(19) United States
(12) Patent Application Publication
(10) Pub. No.: US 2020/0245976 A1
(43) Pub. Date: Aug. 6, 2020

(54) RETROSPECTIVE IMAGE SAVING FOR ULTRASOUND DIAGNOSTICS

(71) Applicant: Bay Labs, Inc., San Francisco, CA (US)

(72) Inventors: Charles Cadieu, Menlo Park, CA (US); Charles Chen, San Mateo, CA (US); Ha Hong, San Ramon, CA (US); Ching Hsieh, San Francisco, CA (US); Kilian Koepsell, San Francisco, CA (US)

(21) Appl. No.: 16/264,151

(22) Filed: Jan. 31, 2019

(51) Int. Cl. A61B 8/08; G16H 30/20; G01S 15/89

(52) U.S. Cl. CPC A61B 8/5207; G16H 30/40; G01S 15/89; G16H 30/20

(57) ABSTRACT

In a method, system and computer program product for retrospective image clip saving for ultrasound imagery, the method includes first repeatedly acquiring different ultrasound image clips of a single view of a target feature through an ultrasound imaging probe of an ultrasound imaging system. Thereafter, each of the acquired different ultrasound image clips may be stored in a buffer of the ultrasound imaging system and a value assigned to each of the image clips according to a measured characteristic. A save-best-clip mode of the ultrasound imaging system is then activated and, in response to the activation, each of the acquired different ultrasound image clips are compared in the buffer based upon respective measured characteristics, at least one of the clips demonstrating an optimal value for the measured characteristic is identified and the identified clip or clips saved in memory of the ultrasound imaging system in correspondence with the single view.

### BACKGROUND OF THE INVENTION

### Field of the Invention

The present invention relates to ultrasound imaging and more particularly to ultrasound image acquisition.

### Description of the Related Art

Ultrasound imaging, also known as sonography, is a medical imaging technique that employs high-frequency sound waves to view three-dimensional structures inside the body of a living being. Because ultrasound images are captured in real-time, ultrasound images also show movement of the internal organs of the body as well as blood flowing through the blood vessels of the human body and the stiffness of tissue. Unlike x-ray imaging, ultrasound imaging does not involve ionizing radiation thereby allowing prolonged usage of ultrasound imaging without threatening tissue and internal organ damage from prolonged radiation exposure.

To acquire ultrasound imagery, during an ultrasound exam, a transducer, commonly referred to as a probe, is placed directly on the skin or inside a body opening. A thin layer of gel is applied to the skin so that the ultrasound waves are transmitted from the transducer through the medium of the gel into the body. The ultrasound image is produced based upon a measurement of the reflection of the ultrasound waves off the body structures. The strength of the ultrasound signal, measured as the amplitude of the detected sound wave reflection, and the time taken for the sound wave to travel through the body provide the information necessary to compute an image.

Compared to other prominent methods of medical imaging, ultrasound presents several advantages to the diagnostician and patient. First and foremost, ultrasound imaging provides images in real-time. As well, ultrasound imaging requires equipment that is portable and can be brought to the bedside of the patient. Further, as a practical matter, the ultrasound imaging equipment is substantially lower in cost than other medical imaging equipment, and as noted, does not use harmful ionizing radiation. Even still, the production of quality ultrasound images remains highly dependent upon a skilled operator.

Of note, during ultrasound imaging, images are captured in real-time and a very high rate of image acquisition. After some manipulation of the imaging probe, ultimately, a suitable set of imagery—literally an image clip—is acquired. However, the duration of time during which the desired image clip is present is limited and, depending upon the movement of the imaging probe, the desired image clip may no longer exist. Consequently, the operator must be quick to recognize the acquisition of the desired image clip and direct the imaging system to store the desired image clip. However, the lapse of time between when the operator recognizes the acquisition of the desired image clip and the moment at which the operator is able to direct the imaging system to store the desired image clip can be enough that the desired image clip is no longer present at the time of directing the imaging system to store the desired image clip. Indeed, the very act of the operator reaching for a control of the imaging system to direct the storage of the desired image clip can cause unwanted movement of the imaging probe so as to degrade the acquired imagery beyond suitable.

### BRIEF SUMMARY OF THE INVENTION

Embodiments of the present invention address deficiencies of the art in respect to image clip acquisition in ultrasound imagery and provide a novel and non-obvious method, system and computer program product for retrospective image clip saving for ultrasound imagery. In an embodiment of the invention, a method for retrospective image clip saving includes first repeatedly acquiring different ultrasound image clips of a single view of a target feature through an ultrasound imaging probe of an ultrasound imaging system. Thereafter, each of the acquired different ultrasound image clips may be stored in a buffer of the ultrasound imaging system and a value assigned to each of the image clips according to a measured characteristic of a corresponding one of the image clips, such as a quality measured for each of the image clips, a quality measured for each of the image clips in respect to a particular disease specified for diagnosis in the ultrasound imaging system, or a number of anatomical structures of the target feature that are visible in a corresponding one of the image clips.

A save-best-clip mode of the ultrasound imaging system is then activated in which one or more previously acquired clips are saved as the most optimal clip or clips acquired in respect to a target feature and specified view, or a detected aspect of a target feature. In response to the activation, each of the acquired different ultrasound image clips are compared in the buffer based upon respective measured characteristics, at least one of the acquired different ultrasound image clips may then be identified that demonstrates an optimal value for the measured characteristic. Finally, the identified one or more of the acquired different ultrasound image clips is saved in the memory of the ultrasound imaging system in correspondence with the single view.

In one aspect of the embodiment, the method additionally includes setting a threshold period of time over which the acquired different ultrasound image clips are to be acquired, initiating a timer upon acquiring a first one of the acquired different ultrasound image clips and performing the activating of the save-best-clip mode subsequent to the timer surpassing the threshold period of time. In another aspect of the embodiment, in response to the saving in memory of the ultrasound imaging system, of the highest quality one or more of the acquired different ultrasound image clips in correspondence with the single view, all the acquired different ultrasound image clips are erased in the buffer, new ultrasound image clips are repeatedly acquired of the single view of the target feature through the ultrasound imaging probe of the ultrasound imaging system, each are then stored in the buffer of the ultrasound imaging system and the save-best-clip mode activated for the acquired new ultrasound image clips in the buffer.

In another aspect of the embodiment, the identifying of the one or more of the acquired different ultrasound image clips includes identifying one of the different ones of the acquired different ultrasound image clips of minimum quality for the single view having a longest duration. To that end, during the storing of each of the acquired different ultrasound image clips in the buffer, for each one of the acquired different ultrasound image clips, a corresponding image quality may be rated, and on condition that the corresponding image quality exceeds a minimum quality for the single view a clip duration of a corresponding one of the acquired different image clips is computed, the computed clip duration compared to a previously stored clip duration of a different one of the acquired different ultrasound image clips having an image quality that exceeds the minimum quality and the corresponding one of the acquired different ultrasound image clips labeled in the buffer as the highest quality one of the acquired different ultrasound image clips on condition that the computed clip duration exceeds the previously stored clip duration.

In another embodiment of the invention, a data processing system is configured for retrospective image clip saving for ultrasound imagery. The system includes a computer with memory and at least one processor, a display coupled to the computer, beamformer circuitry coupled to the computer and the display, and an ultrasound imaging probe that includes a transducer connected to the beamformer circuitry. The system also includes a save-best-clip mode module executing in the memory of the computer. The module includes program code enabled upon execution by the processor of the computer to perform the repeated acquisition of different ultrasound image clips of a single view of a target feature through the ultrasound imaging probe, to store each of the acquired different ultrasound image clips in a buffer of the computer, and to assign a value to each of the image clips according to a measured characteristic of a corresponding one of the image clips, to activate a save-best-clip mode and, in response to the activation of the save-best-clip mode, to compare each of the acquired different ultrasound image clips in the buffer based upon respective measured characteristics, identifying at least one of the acquired different ultrasound image clips demonstrating an optimal value for the measured characteristic, identify one of the acquired different ultrasound image clips identified that demonstrates an optimal value for the measured characteristic and save in the memory the identified one or more of the acquired different ultrasound image clips in correspondence with the single view.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is pictorial illustration of a process for retrospective image clip saving for ultrasound imagery;

FIG. 2 is a schematic illustration of a data processing system configured for retrospective image clip saving for ultrasound imagery; and,

FIG. 3 is a flow chart illustrating a process for retrospective image clip saving for ultrasound imagery.

### DETAILED DESCRIPTION OF THE INVENTION

Embodiments of the invention provide for retrospective image clip saving for ultrasound imagery. In accordance with an embodiment of the invention, ultrasound imagery is continuously acquired by an ultrasound imaging probe of an ultrasound imaging system. As the imagery is acquired, the imagery is displayed in a display of the ultrasound. Concurrently, the imagery is placed in buffer memory of the ultrasound imaging system and indexed as a clip in a sequence of clips stored in the buffer memory. Further, a value is assigned to each of the imagery according to a measured characteristic of a corresponding one of the imagery. Thereafter, a save-best-clip operation is invoked either by way of a lapse of a predetermined period of time, or at the direction of the operator of the ultrasound imaging probe. In response to the invocation of the save-best-clip operation, each of the image clips in the buffer memory are compared with one another based upon respective measured characteristics, to identify a most suitable image clip. The most suitable image clip is then saved as if the operator of the ultrasound imaging probe had manually directed the saving of the most suitable clip when the most suitable clip had been displayed in a display of the ultrasound imaging system.

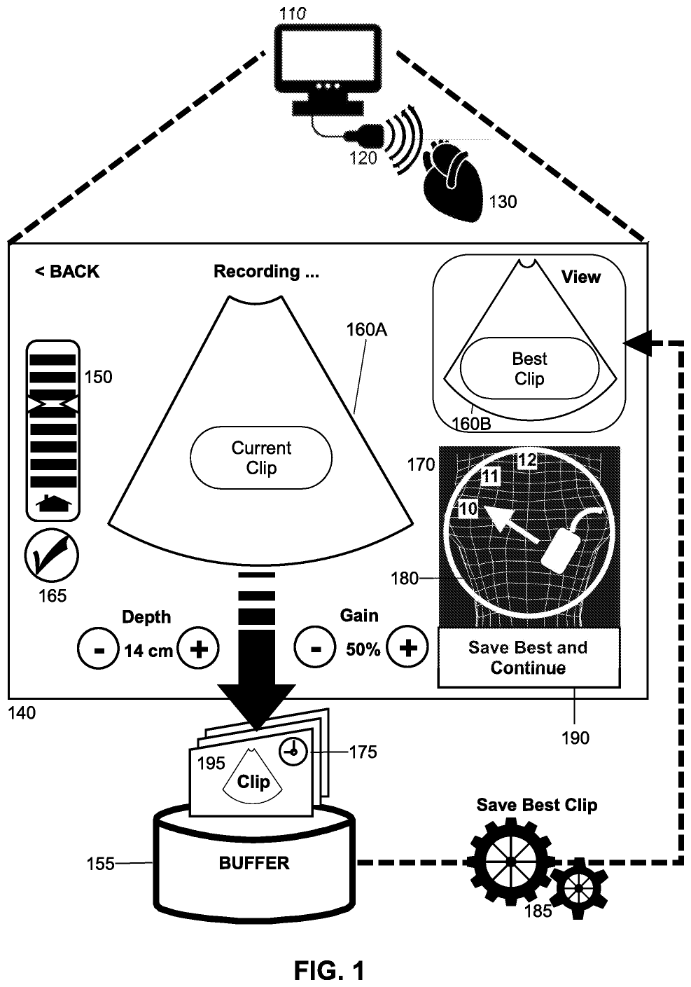

In further illustration, FIG. 1 pictorially shows a process for retrospective image clip saving for ultrasound imagery. As shown in FIG. 1, an ultrasound imaging system 110 with ultrasound imaging probe 120 conducts an ultrasound imaging operation in order to image a target organ 130. A user interface 140 to the ultrasound imaging system 110 presents a contemporaneous display 160A of a video clip acquired by the ultrasound imaging probe 120 of the target organ 130. An acquisition meter 150 is disposed in the user interface 140 and indicates a sliding scale of success of acquiring the video clip in the contemporaneous display 160A relative to a known view sought to be acquired for the target organ 130. For example, in connection with the imaging of a heart, the known view may include a parasternal long axis view, a parasternal short axis view, an apical two, three, four or five chamber view or a subcostal view. To the extent that the video clip in the contemporaneous display is determined to meet or exceed a threshold quality for the view, a success icon 165 is displayed in connection with the acquisition meter 150. To that end, each acquired video clip can be evaluated according to one or more criteria such as quality, the presence of a particular anatomical feature or likelihood that a particular pathology is visible in the video clip. The acquisition meter 150 itself indicates a level of the criteria detected for each contemporaneous video clip and a pre-set threshold value for the criteria.

Of note, during the acquisition of video clip imagery by the ultrasound imaging probe 120, a succession of video clips 195 are acquired and temporarily stored in a buffer 155 of the ultrasound diagnostic system 110. Each of the buffered video clips 195 can include therewith, an indication of a duration of time 175 of the corresponding one of the buffered video clips 195. As well, a quality of each of the buffered video clips 195 may be determined, for instance, by submitting each of the buffered video clips 195 to a neural network trained to determine a characteristic of the video clip, for instance, a quality of a video clip for a specified view of a target organ, or by comparing each of the buffered video clips 195 to a set of known video clips of known characteristics, such as quality, during a content-based image retrieval operation. A portion of the user interface 140 can include a window displaying one 160B of the buffered video clips 195 acquired amongst the buffered video clips 195 that has a measured characteristic such as quality which exceeds that of all others of the buffered video clips 195.

Optionally, an additional portion 170 of the user interface 140 may be provided displaying an iconic image of a portion of a body in which the target organ 130 resides and a recommended movement of the ultrasound imaging probe 120 relative to the iconic image in order to achieve the selected view for the target organ 130. The additional portion of the user interface 170 includes a spatial orientation indicator 180 superimposed upon the iconic image of the portion of the body in which the target organ 130 resides. arranged as a clock angle indicator with twelve angularly equidistant positions. In this way, the combination of the recommended movement and the spatial orientation indicator 180 provide quick visual guidance to the operator of the ultrasound imaging system 110 in order to achieve a video clip of sufficient quality for the selected view.

Importantly, a save-best-clip mode may be activated, either automatically in response to an elapsed time beyond a threshold time from which the attempt to acquire a video clip presenting a characteristic considered satisfactory in respect to a predetermined criteria, for example a video clip of satisfactory quality for the selected view of the target organ 130 began, or in response to a manual selection of a user interface control 190. Once activated, the save-best-clip mode reviews the buffered video clips 195 in the buffer and selects for saving, one of the buffered video clips 195 having the highest associated quality for the selected view of the target organ 130. Optionally, instead of merely saving the highest quality one of the buffered video clips 195, a subset of the buffered video clips 195 may be selected, each exceeding a minimally acceptable quality for the selected view of the target organ 130. Then, amongst the subset, one of the buffered video clips 195 having associated therewith a longest duration is selected as the best clip and saved. In either case, the selected one of the buffered video clips 195 is presented in the window displaying a highest quality one 160B of the buffered video clips 195 acquired amongst the buffered video clips 195. Finally, the buffer 155 is then cleared of all of the buffered video clips 195.

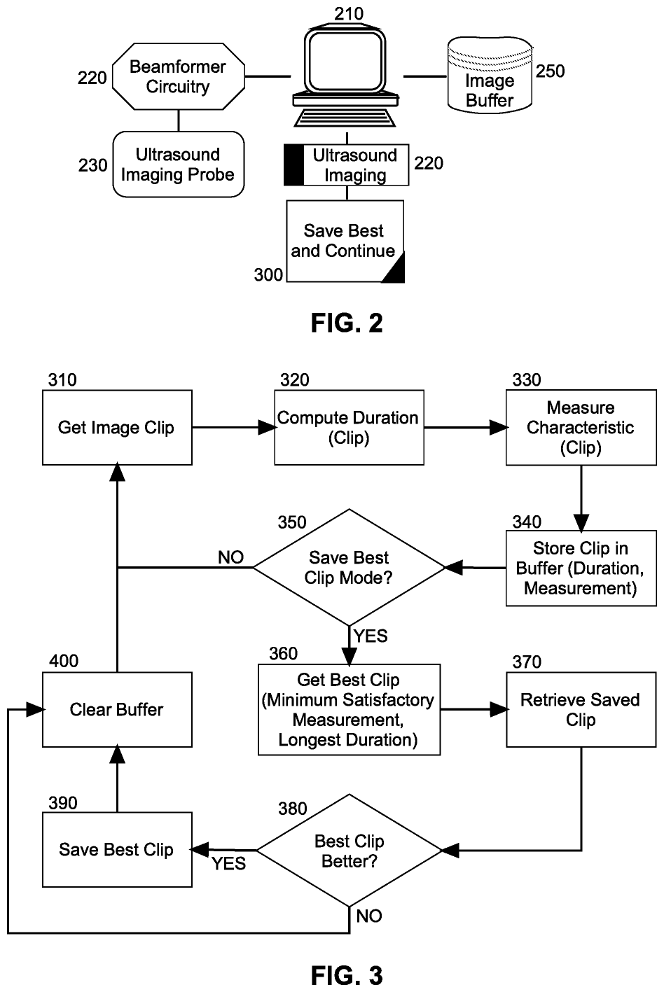

The process described in connection with FIG. 1 may be implemented in a ultrasound diagnostics data processing system. In further illustration, FIG. 2 schematically shows a data processing system configured for retrospective image clip saving for ultrasound imagery. The system includes a host computing system 210 that includes a computer with at least one processor, memory and a display. The host computing system 210 also includes a buffer 250. The host computing system 210 yet further is coupled to an ultrasound imaging system 220 adapted to store in the buffer 250 ultrasound imagery acquired through the placement of an imaging wand 230 proximate to a target organ of interest in a mammalian subject by operation of beamformer circuitry 220.

Importantly, the host computing system 210 is communicatively coupled to fixed storage (not shown), either locally or remotely (“in the cloud”) storing therein a neural network and a programmatic interface to the neural network. The neural network is trained to characterize one or more features of the target organ, for example an ejection fraction value of a heart, or the presence or absence of aortic stenosis. To do so, video clip imagery of a specified view of the target organ acquired by the ultrasound imaging system 220 is provided to the neural network which in turn accesses the programmatic interface so that the neural network may then output the characterization for the video clip imagery along with an indication of confidence in that characterization. The ultrasound imaging system 220 in turn renders on the display of the host computing system 210 not only the video clip imagery, but also the characterization and optionally, the indication of confidence.

In accordance with an embodiment of the invention, a save best video clip module 300 is included with the ultrasound imaging application 220. The module 300 includes computer program instructions enabled, upon execution in the host computing system 210, to store successively acquired video clips of the target organ in the buffer 250, assigning each a quality and a duration value. Then, upon activation of a save best video clip mode, a video clip of the highest relative quality to other video clips in the buffer 250 for the specified view of the target organ is displayed in a portion of the user interface to the ultrasound imaging system 220 and stored to fixed storage as the video clip of best quality acquired during imaging of the target organ for the specified view. Thereafter, the video clips in the buffer 250 are deleted and the process may continue for imaging the target organ for a different view.

In even further illustration of the operation of the save best video clip module 300, FIG. 3 is a flow chart illustrating a process for retrospective image clip saving for ultrasound imagery. Beginning in block 310, a video clip of a target organ according to a specified view is received in memory of the host computing system supporting the operation of the ultrasound imaging system. In block 320, a duration of the received video clip is computed and in block 330, a quality of the received video clip in respect to the specified view is determined. Then, in block 340, the received video clip is stored temporarily in a buffer along with the quality indication and duration.

In decision block 350, it is determined if the save best video clip mode has been activated. If not, the process can return to block 310 with the acquisition of a next video clip of the target organ according to the specified view and the buffering of the next video clip of the target organ along with a corresponding indication of quality and duration. In decision block 350, if it is determined that the save best video clip mode has been activated, in block 360, a best one of the buffered video clips in the buffer is selected that has a minimum quality beyond a threshold quality for the specified view and a longest duration. The selected video clip is then retrieved from the buffer in block 370 and compared to a prior best clip selected from amongst a previous selection of buffered video clips. In decision block 380, if it is determined that the selected video clip exceeds in quality or duration or both, that of the prior best clip, in block 390, the selected video clip is saved in place of the prior best clip and in block 400, the buffer is cleared. Thereafter, the process returns to block 310.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Computer readable program instructions described herein can be downloaded to respective computing/processing devices from a computer readable storage medium or to an external computer or external storage device via a network. The computer readable program instructions may execute entirely on the user's computer, partly on the user's computer, as a stand-alone software package, partly on the user's computer and partly on a remote computer or entirely on the remote computer or server. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Finally, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

We claim:

1. A method for retrospective image clip saving for ultrasound imagery, the method comprising:
repeatedly acquiring different ultrasound image clips of a single view of a target feature through an ultrasound imaging probe of an ultrasound imaging system;
storing each of the acquired different ultrasound image clips in a buffer of the ultrasound imaging system;
assigning a value to each of the image clips according to a measured characteristic of a corresponding one of the image clips;
activating a save-best-clip mode of the ultrasound imaging system; and,
responsive to the activation of the save-best-clip mode, comparing each of the acquired different ultrasound image clips in the buffer based upon respective measured characteristics, identifying at least one of the acquired different ultrasound image clips demonstrating an optimal value for the measured characteristic and saving in memory of the ultrasound imaging system, the identified at least one of the acquired different ultrasound image clips in correspondence with the single view.

2. The method of claim 1, further comprising:
setting a threshold period of time over which the acquired different ultrasound image clips are to be acquired;
initiating a timer upon acquiring a first one of the acquired different ultrasound image clips; and,
performing the activating of the save-best-clip mode subsequent to the timer surpassing the threshold period of time.

3. The method of claim 1, wherein the identifying of the at least one of the acquired different ultrasound image clips comprises identifying one of the different ones of the acquired different ultrasound image clips of minimum quality for the single view having a longest duration.

4. The method of claim 3 wherein during the storing of each of the acquired different ultrasound image clips in the buffer, for each one of the acquired different ultrasound image clips:
rating a corresponding image quality, and,
on condition that the corresponding image quality exceeds a minimum quality for the single view:
computing a clip duration of a corresponding one of the acquired different image clips,
comparing the computed clip duration to a previously stored clip duration of a different one of the acquired different ultrasound image clips having an image quality that exceeds the minimum quality, and
labeling in the buffer the corresponding one of the acquired different ultrasound image clips as the highest quality one of the acquired different ultrasound image clips on condition that the computed clip duration exceeds the previously stored clip duration.

5. The method of claim 1, further comprising:
responsive to the saving in memory of the ultrasound imaging system, of the identified at least one of the acquired different ultrasound image clips in correspondence with the single view, erasing all the acquired different ultrasound image clips in the buffer, repeatedly acquiring new ultrasound image clips of the single view of the target feature through the ultrasound imaging probe of the ultrasound imaging system, storing each of the acquired new ultrasound image clips in the buffer of the ultrasound imaging system and activating the save-best-clip mode for the acquired new ultrasound image clips in the buffer.

6. The method of claim 1, wherein the measured characteristic is a highest measured quality of each of the image clips in respect to a particular disease specified for diagnosis in the ultrasound imaging system.

7. The method of claim 1, wherein the measured characteristic is a number of visible anatomical structures known to be present in the target feature.

8. A data processing system configured for retrospective image clip saving for ultrasound imagery, the system comprising:
a computer with memory and at least one processor;
a display coupled to the computer;
beamformer circuitry coupled to the computer and the display;
an ultrasound imaging probe comprising a transducer connected to the beamformer circuitry; and,
a save-base-clip mode module executing in the memory of the computer, the module comprising program code enabled upon execution by the processor of the computer to perform:
repeatedly acquiring different ultrasound image clips of a single view of a target feature through the ultrasound imaging probe;
storing each of the acquired different ultrasound image clips in a buffer of the computer;
assigning a value to each of the image clips according to a measured characteristic of a corresponding one of the image clips;
activating a save-best-clip mode; and,
responsive to the activation of the save-best-clip mode, comparing each of the acquired different ultrasound image clips in the buffer based upon respective measured characteristics, identifying at least one of the acquired different ultrasound image clips demonstrating an optimal value for the measured characteristic and saving in memory of the ultrasound imaging system, the identified at least one of the acquired different ultrasound image clips in correspondence with the single view, identifying a highest quality one of the acquired different ultrasound image clips and saving in the memory the highest quality one of the acquired different ultrasound image clips in correspondence with the single view.

9. The system of claim 8, wherein the program code of the module is further enabled to perform:
setting a threshold period of time over which the acquired different ultrasound image clips are to be acquired;
initiating a timer upon acquiring a first one of the acquired different ultrasound image clips; and,
performing the activating of the save-best-clip mode subsequent to the timer surpassing the threshold period of time.

10. The system of claim 8, wherein the identifying of the at least one of the acquired different ultrasound image clips comprises identifying one of the different ones of the acquired different ultrasound image clips of minimum quality for the single view having a longest duration.

11. The system of claim 10, wherein during the storing of each of the acquired different ultrasound image clips in the buffer, for each one of the acquired different ultrasound image clips, the program code of the module performs:
rating a corresponding image quality, and,
on condition that the corresponding image quality exceeds a minimum quality for the single view:
computing a clip duration of a corresponding one of the acquired different image clips,
comparing the computed clip duration to a previously stored clip duration of a different one of the acquired different ultrasound image clips having an image quality that exceeds the minimum quality, and
labeling in the buffer the corresponding one of the acquired different ultrasound image clips as the highest quality one of the acquired different ultrasound image clips on condition that the computed clip duration exceeds the previously stored clip duration.

12. The system of claim 8, wherein the program code of the module further performs:
responsive to the saving in memory the at least one of the acquired different ultrasound image clips in correspondence with the single view, erasing all the acquired different ultrasound image clips in the buffer, repeatedly acquiring new ultrasound image clips of the single view of the target feature through the ultrasound imaging probe, storing each of the acquired new ultrasound image clips in the buffer and activating the save-best-clip mode for the acquired new ultrasound image clips in the buffer.

13. The system of claim 8, wherein the measured characteristic is a number of visible anatomical structures known to be present in the target feature.

14. A computer program product for retrospective image clip saving for ultrasound imagery, the computer program product including a computer readable storage medium having program instructions embodied therewith, the program instructions executable by a device to cause the device to perform a method including:
repeatedly acquiring different ultrasound image clips of a single view of a target feature through an ultrasound imaging probe of an ultrasound imaging system;
storing each of the acquired different ultrasound image clips in a buffer of the ultrasound imaging system;
assigning a value to each of the image clips according to a measured characteristic of a corresponding one of the image clips;
activating a save-best-clip mode of the ultrasound imaging system; and,
responsive to the activation of the save-best-clip mode, comparing each of the acquired different ultrasound image clips in the buffer based upon respective measured characteristics, identifying at least one of the acquired different ultrasound image clips demonstrating an optimal value for the measured characteristic and saving in memory of the ultrasound imaging system, the identified at least one of the acquired different ultrasound image clips in correspondence with the single view, identifying a highest quality one of the acquired different ultrasound image clips and saving in memory of the ultrasound imaging system, the highest quality one of the acquired different ultrasound image clips in correspondence with the single view.

15. The computer program product of claim 14, wherein the method further comprises:
setting a threshold period of time over which the acquired different ultrasound image clips are to be acquired;
initiating a timer upon acquiring a first one of the acquired different ultrasound image clips; and,
performing the activating of the save-best-clip mode subsequent to the timer surpassing the threshold period of time.

16. The computer program product of claim 14, wherein the identifying of the at least one of the acquired different ultrasound image clips comprises identifying one of the different ones of the acquired different ultrasound image clips of minimum quality for the single view having a longest duration.

17. The computer program product of claim 16 wherein during the storing of each of the acquired different ultrasound image clips in the buffer, for each one of the acquired different ultrasound image clips, the method further comprises:
rating a corresponding image quality, and,
on condition that the corresponding image quality exceeds a minimum quality for the single view:
computing a clip duration of a corresponding one of the acquired different image clips,
comparing the computed clip duration to a previously stored clip duration of a different one of the acquired different ultrasound image clips having an image quality that exceeds the minimum quality, and
labeling in the buffer the corresponding one of the acquired different ultrasound image clips as the highest quality one of the acquired different ultrasound image clips on condition that the computed clip duration exceeds the previously stored clip duration.

18. The system of claim 14, wherein the method further comprises:
responsive to the saving in memory of the ultrasound imaging system, of the at least one of the acquired different ultrasound image clips in correspondence with the single view, erasing all the acquired different ultrasound image clips in the buffer, repeatedly acquiring new ultrasound image clips of the single view of the target feature through the ultrasound imaging probe of the ultrasound imaging system, storing each of the acquired new ultrasound image clips in the buffer of the ultrasound imaging system and activating the save-best-clip mode for the acquired new ultrasound image clips in the buffer.

19. The computer program of claim 14, wherein the measured characteristic is a highest measured quality of each of the image clips in respect to a particular disease specified for diagnosis in the ultrasound imaging system.

20. The computer program product of claim 14, wherein the measured characteristic is a number of visible anatomical structures known to be present in the target feature.
